SEQUENCE LISTING (3 sequences)

SEQ ID NO: 1 - protein, 12 aa, synthetic construct
  REGION 1..12: note = Synthetic
WKGWSYLWTQQA

SEQ ID NO: 2 - protein, 5 aa, synthetic construct
  REGION 1..5: note = Synthetic
GGGSK

SEQ ID NO: 3 - protein, 12 aa, synthetic construct
  REGION 1..12: note = Synthetic
WYKAQQWWTLGS

(19) United States
(12) Patent Application Publication
(10) Pub. No.: US 2025/0073355 A1
(43) Pub. Date: Mar. 6, 2025

(54) CD44-BINDING PEPTIDE REAGENTS AND METHODS

(71) Applicant: THE REGENTS OF THE UNIVERSITY OF MICHIGAN, Ann Arbor, MI (US)

(72) Inventors: Thomas D. Wang, Ann Arbor, MI (US); Xiaoqing Meng, Ann Arbor, MI (US)

(21) Appl. No.: 18/708,809

(22) PCT Filed: Nov. 11, 2022
(86) PCT No.: PCT/US2022/079754

§ 371 Date: May 9, 2024

Related U.S. Application Data
(60) Provisional application No. 63/278,880, filed on Nov. 12, 2021.

(51) Int. Cl. A61K 49/00; C07K 7/08

(52) U.S. Cl. CPC A61K 49/0056; A61K 49/0021; C07K 7/08; C07K 2319/60

(57) ABSTRACT

The present disclosure relates to CD44-binding peptide reagents, methods for detecting cells such as hepatocellular carcinoma cells using the peptide reagents, and methods for targeting such cells using the peptide reagents.

### CROSS-REFERENCE TO RELATED APPLICATIONS

This application claims priority to U.S. Provisional Patent Application No. 63/278,880, filed on Nov. 12, 2021, which is incorporated by reference herein in its entirety.

### STATEMENT OF GOVERNMENT SUPPORT

This invention was made with government support under Grant No. U01 CA230669 awarded by the National Institute of Health. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 57043A_Seqlisting.XML; Size: 4,554 bytes; Created: Nov. 8, 2022.

### FIELD OF THE INVENTION

The present disclosure relates to CD44-binding peptide reagents, methods for detecting hepatocellular carcinoma cells using the peptide reagents, and methods for targeting such cells using the peptide reagents.

### BACKGROUND

Hepatocellular carcinoma (HCC) accounts for over 840,000 deaths globally, and is emerging rapidly as a major contributor to the worldwide healthcare burden. Because few patients are diagnosed early, 5-year survival is <7%, and the median survival length is <1 year [Asrani et al., Burden of liver diseases in the world, 70(1) (2019) 151-171.]. In the U.S., the incidence of HCC is rising steadily, and is currently growing faster than any other cancer [Ozakyol, Global Epidemiology of Hepatocellular Carcinoma (HCC Epidemiology). J Gastrointest Cancer 2017; 48:238-2407]. Conventional methods for liver imaging excel at providing anatomical features of masses. Ultrasound is recommended for patients with cirrhosis, but cannot distinguish between malignant and benign lesions. Contrast-enhanced CT and MRI detect HCC based on increased vascularity, but cannot clarify pathology for liver nodules<1-2 cm. Malignant hepatocytes uniquely overexpress targets that can be developed for improved HCC diagnosis and therapy. Thus, early detection of HCC remains a major healthcare challenge globally, and novel diagnostic options are urgently needed.

Cluster of differentiation 44 (CD44) is a multi-structural and multi-functional cell surface molecule involved in cell proliferation, differentiation, migration, and angiogenesis and in presentation of cytokines, chemokines, and growth factors as well as in cell signaling. Awareness of a connection between CD44 expression and cancer dates back more than two decades. However, CD44 has recently been demonstrated as an universal marker on cancer stem cells/tumor initiating cells (CSCs/TICs) [Naor et al., Critical Reviews in Clinical Laboratory Sciences, 39(6) (2002) 527-579; Ghosh et al., Expert Opinion on Therapeutic Targets 16(7) (2012) 635-650; Bose et al., J Stem Cell Res Ther, 4(173) (2014) 2; Ponta et al., Pediatric Pathology & Molecular Medicine, 18(4-5) (1998) 381-393.]. Recent studies have revealed that the increased CD44 expression in HCC is correlated with increased metastasis, recurrence, resistance to chemotherapy or radiation therapy, and decreased survival [Mima et al., Cancer Research, 72(13) (2012) 3414-3423; Okabe et al., British Journal of Cancer, 110(4) (2014) 958-966; Ji et al., Clinical implications of cancer stem cell biology in hepatocellular carcinoma, Seminars in Oncology, Elsevier, 2012, pp. 461-472].

New products and methods for detection and treatment of hepatocellular carcinoma are needed in the art.

### SUMMARY

In one aspect, the disclosure provides a reagent comprising a peptide WKGWSYLWTQQA (SEQ ID NO: 1), or a multimer form of the peptide, wherein the reagent binds to CD44. The multimer form can be a dimer. The peptide reagent can consist essentially of the peptide or multimer form of the peptide.

The reagent comprises at least one detectable label, at least one therapeutic moiety, or both, attached to the peptide or multimer form of the peptide.

The detectable label can be detected by optical, photoacoustic, ultrasound, positron emission tomography (PET) or magnetic resonance imaging. The label detectable by optical imaging can be fluorescein isothiocyanate (FITC), Cy5, Cy5.5, or IRdye800. The label detectable by magnetic resonance imaging can be gadolinium (Gd) or Gd-DOTA. The detectable label can be attached to the peptide by a peptide linker. The terminal amino acid of the linker can be lysine. The linker can comprise the sequence GGGSC. The linker can comprise the sequence GGGSK set out in SEQ ID NO: 2.

The therapeutic moiety can be a chemopreventative or chemotherapeutic agent such as celecoxib, carboplatin, paclitaxel, cisplatin, 5-fluorouracil (5-FU), oxaliplatin, capecitabine, chlorambucil, sorabenib and irinotecan. The therapeutic moiety can be a nanoparticle or micelle, such as a polymeric nanoparticle or polymeric micelle, encapsulating a chemopreventative or chemotherapeutic agent (including, but not limited to, celecoxib, carboplatin, paclitaxel, cisplatin, 5-fluorouracil (5-FU), oxaliplatin, capecitabine, chlorambucil, sorabenib and irinotecan).

The regent can comprise at least one detectable label attached to the peptide or multimer form of the peptide and at least one therapeutic moiety attached to the peptide or multimer form of the peptide.

In another aspect, the disclosure provides a composition comprising a reagent provided herein and a pharmaceutically acceptable excipient.

In yet another aspect, the disclosure provides methods for detecting HCC cells in a patient comprising the steps of administering a reagent provided herein to the patient and detecting binding of the reagent to cancerous cells.

In another aspect, the disclosure provides a method of determining the effectiveness of a treatment for HCC and/or HCC metastasis, or recurrence of HCC in a patient comprising the step of administering a reagent provided herein to the patient, visualizing a first amount of cells labeled with the reagent, and comparing the first amount to a previously-visualized second amount of cells labeled with the reagent, wherein a decrease in the first amount cells labeled relative to the previously-visualized second amount of cells labeled is indicative of effective treatment. The methods can further comprise obtaining a biopsy of the cells labeled by the reagent.

In yet another aspect, the disclosure provides a method for delivering a therapeutic moiety to HCC cells in a patient comprising the step of administering a reagent provided herein to the patient.

In a further aspect, the disclosure provides a kit for administering a composition of disclosed herein to a patient in need thereof, comprising the composition, instructions for use of the composition and a device for administering the composition to the patient.

In another aspect, the disclosure provides a peptide consisting of the amino acid sequence WKGWSYLWTQQA (SEQ ID NO: 1).

The following Drawings and Detailed Description (including the Examples) illustrate various non-limiting aspects of the subject matter contemplated herein.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 shows a contact map of the interface between an initial candidate peptide sequence and its CD44 target.

FIG. 2 shows pairing frequency for a number of aligned peptide/receptor residues from Table 1.

FIG. 3A-B shows a structural model for CD44. Docking energy for binding of WKG* and WYK* to CD44 was evaluated using a structural model (1 UUH). A) The sequence WKGWSYLWTQQA was found to bind CD44 with a total energy Et=−534. B) This sequence was scrambled as WYKAQQWWTLGS for use as control and resulted in Et=−494.

FIG. 4A-D shows an optimized peptide specific for CD44. A) Peptide WKGWSYLWTQQA (blue) is labeled with an IRDye800 fluorophore (red) via a GGGSC linker (black). B) The sequence is scrambled as WYKAQQWWTLGS for use as control. C,D) 3D models show differences in biochemical structures.

FIG. 5A-B shows mass spectrometry results for peptides. Experimental mass-to-charge ratios (m/z) for A) WKG* and B) WYK* were found to be 1913.87, which agrees with the expected value of 1913.88.

FIG. 6A-B shows spectral properties of peptides. WKG*-IRDye800 and WYK*-IRDye800 were found to have peak A) absorbance at λ_{abs}=775 nm and B) emission at λₑₘ=810 nm, respectively.

FIG. 7A-F shows validation of specific peptide binding. A) WKG*-IRDye800 (red) and anti-CD44-AF488 (green) show strong binding to the surface (arrows) of human SK-Hep1 HCC cells transfected with control siRNA (siCL). Co-localization of binding of the two peptides can be appreciated on the merged image. The scrambled control WKG*-IRDye800 shows minimal binding. B-D) The fluorescence intensities measured for WKG*-IRDye800 and anti-CD44-AF488 are greatly reduced with CD44 knockdown using three different siRNAs. WYK*-IRDye800 shows little binding to knockdown cells. E) Quantification of fluorescence intensities show a significant difference in intensities for anti-CD44-AF488 (siCL 4.1, 3.3, and 3.1-fold change in intensity relative to siCD441, siCD442, siCD443) and WKG* IRDye800 (4.0, 3.1, 3.1-fold change). WYK* IRDye800 shows no significant differences. F) Western blot shows CD44 expression for control (siCL) and knockdown (siCD44) cells.

FIG. 8A-C shows binding co-localization. A) WKG*-IRDye800 and B) anti-CD44-AF488 bind to the surface (arrows) of SK-Hep1 cells. C) A Pearson correlation coefficient of ρ=0.81 was measured on the merged image.

FIG. 9A-C shows peptide binding to HCC cells with different levels of CD44 expression. A) Using confocal microscopy, anti-CD44-AF488 (green) and WKG*-IRDye800 (red) show strong binding to the surface of human SK-Hep1 and Hep 3B HCC cells. Co-localization of binding can be appreciated on the merged image. B) Quantified measurements show that WKG*-IRDye800 and anti-CD44-AF488 have significantly greater intensities than WYK*-IRDye800 (control) with respect to SK-Hep1 binding (4.05 and 5.61-fold change, respectively). No significant differences in intensity were observed for binding to Hep3B cells. C) Western blot shows CD44 expression level for SK-Hep1 and Hep 3B cells.

FIG. 10A-E shows characterization of peptide binding. A) Binding by WKG*-IRDye800 (red) to SK-Hep1 human HCC cells decreases significantly with competition from unlabeled WKG* but not with addition of B) WYK* (control). C) Quantified fluorescence intensities show a concentration-dependent reduction, with significantly lower intensity when unlabeled WKG* is added relative to an equal concentration of WYK* (1.02, 0.61, 0.36, 0.12, and 0.10-fold change, respectively). D) An apparent dissociation constant of k_{d}=43 nM, R²=0.99, is measured for binding of WKG*-IRDye800 to SK-Hep1 cells. E) An apparent association time constant k=0.26 min⁻¹ (6.8 min), R²=0.95, is measured. Results are representative of three independent experiments.

FIG. 11 shows peptide effect on CD44 cell signaling and cell viability. Low molecular weight hyaluronan (HA) at a concentration of 100 μg/mL (positive control) induces phosphorylation of downstream AKT (pAKT) and Erk1/2 (pErk1/2) in SK-Hep1 cells after 15 min of incubation. No HA (none) serves as a negative control. By comparison, incubation with WKG*-IRDye800 at either 4 or 300 μM shows no effect on CD44 downstream signaling. β-actin was used as a loading control.

FIG. 12 shows cell viability. Human SK-Hep1 HCC cells were incubated with peptides at concentrations ranging from 0 to 200 μg/mL for 24 hours. Cytotoxicity was then evaluated using a MTT assay. WKG*-IRDye800 and WYK*-IRDye800 showed decreased cell viability at the highest concentrations.

FIG. 13A-B shows serum stability. A) WKG*-IRDye800 was incubated in mouse serum for 0, 0.5, 1.0, 2, 4, 8, and 24 hours, and serum stability was measured using analytical RP-HPLC. B) The relative concentration was determined by the area-under-the-peak, and a half-life of T_{1/2}=5.1 hours was measured, R²=0.99.

FIG. 14A-E shows in vitro photoacoustic imaging. A) Images of orthotopic human HCC xenograft tumors (SK-Hep1) were collected with excitation at λₑₓ=774 nm before (0 hour) and at 0.5, 1, 1.5, 1.75, 2, 4, and 24 hours after intravenous injection of WKG*-IRDye800. After transient changes, the intensity peaks at 1.75 hours. Photoacoustic images are shown for unlabeled WKG* was injected 20 min prior to WKG*-IRDye800 to compete for binding (block), WYK*-IRDye800, and ICG. B) MRI images confirm orthotopic location of the HCC tumors (arrows). C) Representative 3D photoacoustic image reconstruction of the tumors is shown at 1.75 hours post-injection with a width 5.4 mm, length 9.4 mm (top view) and depth 4.4 mm (side view). D) The quantified T/B ratio confirms peak uptake of WKG*-IRDye800 by tumor at 1.75 hours. Block and WYK*-IRDye800 displayed reduced signal over 24 hours. The intensity from ICG was low early and increased gradually over 24 hours. E) At 1.75 hours post-injection, the quantified T/B ratio for WKG*-IRDye800 was significantly greater than those of block, WYK*-IRDye800, and ICG (mean±SD: 7.12±0.77, 1.74±0.13, 1.47±0.13, and 1.39±0.13, respectively, n=5 mice were evaluated for each group). The adjacent non-tumor tissue region with equal area to the tumor region was used for background.

FIG. 15A-C shows in vitro whole body fluorescence imaging. A) Whole body fluorescence images were collected with excitation at λₑₓ=800 nm before (0 hour) and at 0.5, 1, 1.5, 1.75, 2, 4, and 24 hours after intravenous injection of WKG*-IRDye800. Unlabeled WKG*, injected 20 min prior to WKG*-IRDye800 to compete for binding (block), and WYK*-IRDye800 showed reduced values over 24 hours. The result for ICG (control) was low initially, but increased over time. Peak signals at 1.75 hours from the site of the tumors (circle) support the photoacoustic results. B) The quantified T/B ratio confirms a peak uptake of WKG*-IRDye800 by tumor at 1.75 hours. The adjacent non-tumor tissue region with equal area to the tumor region was used for background. C) The quantified T/B ratio for WKG*-IRDye800 was significantly greater than those of block, WYK*-IRDye800, and ICG (mean±SD: 6.42±0.69, 1.09±0.21, 1.85±0.30, and 0.46±0.03, respectively, n=5 mice were evaluated for each group). The adjacent non-tumor liver tissue region with equal area to the tumor region was used for background.

FIG. 16A-K shows in vitro laparoscopic imaging. Representative A) ultrasound (US) and B) T₁-weighted MR images (MRI) confirm orthotopic location of human HCC xenograft tumors (arrows). Representative white light (WL) and fluorescence (FL) images collected in vivo are shown at 1.75 hours post-injection of C) WKG*-IRDye800, D) WKG* (block), E) WYK*-IRDye800, and F) ICG. G) The quantified T/B ratio for WKG*-IRDye800 was significantly greater than those of block, WYK*-IRDye800, and ICG (mean±SD: 2.32±0.44, 1.13±0.15, 1.21±0.17, and 0.87±0.2, respectively, n=8 mice were evaluated in each group). Background was defined as the adjacent non-tumor region with equal area of the tumor region. Immunohistochemistry (IHC) using H) human-specific anti-cytokeratin and I) anti-CD44 shows presence of HCC xenograft tumor (arrow) adjacent to mouse liver (arrowhead) to confirm orthotopic location. J) Immunofluorescence (IF) of adjacent section supports this result. K) Histology (H&E) from adjacent section is shown.

FIG. 17A-E shows peptide biodistribution. Representative fluorescence images are shown from major organs. The mice were euthanized 1.75 hours after intravenous injection of A) WKG*-IRDye800, B) block, C) WYK*-IRDye800, and D) ICG, n=5 mice per group. E) Quantified results showed that uptake of WKG*-IRDye800 was significantly higher in tumors versus block, WYK*, and ICG (mean±SD: 2.91±0.17, 1.36±0.09, 1.46±0.23, and 1.65±0.24, respectively). The WKG*-IRDye800 intensity was significantly greater in the tumor region than in the adjacent normal liver region (mean±SD: 2.91±0.17, 0.92±0.45).

FIG. 18A-B shows animal necropsy. A) Mice were sacrificed 48 hours post-injection with WKG*-IRDye800. No signs of acute toxicity were seen on histology (H&E) of vital organs, including heart, liver, spleen, lung, kidney, stomach, intestine and brain, and from B) hematology. Results shown represent mean values collected from n=3 mice.

FIG. 19A-G shows specific peptide binding to human HCC ex vivo. A) WKG*-IRDye800 (red) and anti-CD44-AF488 (green) show strong binding to the cell surface (arrows) of HCC using immunofluorescence. B) Diffuse signal is observed for cirrhosis. C) Mild staining is seen with peptide and antibody to hepatic adenoma. D) Minimal intensity is seen for normal human liver. E) Quantified fluorescence intensities show that the intensities associated with HCC is significantly greater than those for adenoma, cirrhosis, and normal human liver (mean±SD: 1.47±0.50, 0.93±0.35, 0.67±0.34, and 0.56±0.21, n=86 human specimens were evaluated). F) ROC curve shows 87% sensitivity and 69% specificity for WKG*-IRDye800 to distinguish HCC from cirrhosis with an AUC=0.79. G) ROC curve shows 87% sensitivity and 79% specificity to distinguish HCC from non-HCC with AUC=0.87.

FIG. 20A-D shows cell-derived hepatocellular carcinoma (HCC) xenograft tumors implanted orthotopically in mice. A) Ultrasound (US), B) MRI (9.4T scanner), and C) laparoscopy in the live mice confirmed the orthotopic location of the HCC tumors. D) The liver was evaluated using immunohistochemistry (IHC), and increased anti-cytokeratin reactivity confirmed the presence of human HCC tumor tissues proliferating within mouse liver.

FIG. 21A-E shows patient-derived xenograft (PDTX) HCC tumors implanted orthotopically in mice. A) Laparoscopic image showed a viable human HCC tumor implanted in mouse liver. B) T₁-weighted MRI image shows orthotopic PDTX HCC tumor at 1.5 hours post-injection of lead CD44 peptide labeled with Gd-DOTA. A target-to-background (T/B) ratio of 2.68 was measured from the PDTX HCC tumor. C-E) Immunohistochemistry (IHC) of PDTX HCC tumors show strong staining (arrows) for GPC3, CD44, and EpCAM, respectively.

FIG. 22 shows an optimized peptide specific for CD44. In the figure, the peptide WKGWSYLWTQQA (black) is labeled with Gd-DOTA (gold) via a GGGSK linker (blue).

### DETAILED DESCRIPTION

Unless otherwise defined herein, scientific and technical terms used herein have the meanings that are commonly understood by those of ordinary skill in the art to which the claimed subject matter belongs.

Image-guided surgery that targets overexpression of molecules that are specific for HCC can help achieve a balance between complete tumor resection and maintenance of tissue function. Targeted imaging can also help maximize the remaining volume of “normal” tissue to optimize post-operative function. In addition, imaging targets specific for HCC can serve as important biomarkers for evaluating patient prognosis. Imaging reagents can provide a biological basis for disease detection, prognosis, guide therapy, and monitor treatment response. Antibodies have been most commonly used, however they are large in size, high in molecular weight, and have long plasma half-lives, all leading to increased background on imaging. Peptides are attractive imaging tools, with a small size and low molecular weight that result in improved properties for deep tissue imaging inaccessible to antibodies. Peptides are less immunogenic, clear from non-target tissues to reduce background, and can be synthesized for improved binding affinity. All of this promotes deep tissue penetration and effective targeting.

In one aspect, the disclosure provides peptides that bind to CD44 expressed on HCC cells. The peptides include, but are not limited to, the peptide WKGWSYLWTQQA (SEQ ID NO: 1).

In a further aspect, the disclosure provides reagents comprising a peptide provided herein. A “peptide reagent” comprises at least two components, a peptide provided herein and another moiety attached to the peptide. The only component of the reagent that contributes to binding of CD44 is the CD44-binding peptide. In other words, the reagent “consists essentially of” a peptide provided herein. The other moiety can comprise amino acids, but the peptide provided herein is not linked to those amino acids in nature and the other amino acids do not affect binding of the peptide to CD44. Moreover, the other moiety in a reagent contemplated herein is not a phage in a phage display library or a component of any other type of peptide display library.

The reagents can comprise at least one detectable label as a moiety attached to a peptide provided herein. The detectable label can be detected, for example, by optical, ultrasound, PET, SPECT, or magnetic resonance imaging. The label detectable by optical imaging can be fluorescein isothiocyanate (FITC), Cy5, Cy5.5 or IRdye800 (also known as IR8000W).

The detectable label can be attached to a peptide provided herein by a peptide linker. The terminal amino acid of the linker can be a lysine such as in the exemplary linker GGGSK (SEQ ID NO: 2) or a cysteine such as in the exemplary linker GGGSC.

The reagents comprise at least one therapeutic moiety attached to a peptide provided herein. The therapeutic moiety can be a chemopreventative or chemotherapeutic agent. The chemopreventative agent can be celecoxib. The chemotherapeutic agent can be carboplatin, paclitaxel, cisplatin, 5-fluorouracil (5-FU), oxaliplatin, capecitabine, chloambucil, sorafenib or irinotecan. The therapeutic moiety can be a nanoparticle or micelle encapsulating another therapeutic moiety. Carboplatin, paclitaxel, cisplatin, 5-fluorouracil (5-FU), oxaliplatin, capecitabine, chloambucil, sorafenib or irinotecan can be encapsulated.

The regent can comprise at least one detectable label attached to the peptide or multimer form of the peptide, and at least one therapeutic moiety attached to the peptide or multimer form of the peptide.

In yet a further aspect, the disclosure provides a composition comprising a reagent provided herein and a pharmaceutically acceptable excipient.

In still a further aspect, the disclosure provides a method for specifically detecting HCC cells in a patient comprising the steps of administering a reagent provided herein attached to a detectable label to the patient and detecting binding of the reagent to the cells.

The detectable binding can take place in vitro, in vitro or in situ.

The phrase “specifically detects” means that the reagent binds to and is detected in association with a type of cell, and the reagent does not bind to and is not detected in association with another type of cell at the level of sensitivity at which the method is carried out.

In an additional aspect, the disclosure provides a method of determining the effectiveness of a treatment for HCC, HCC metastasis, or recurrence of HCC in a patient comprising the step of administering a reagent provided herein attached to a detectable label to the patient, visualizing a first amount of cells labeled with the reagent, and comparing the first amount to a previously-visualized second amount of cells labeled with the reagent, wherein a decrease in the first amount cells labeled relative to the previously-visualized second amount of cells labeled is indicative of effective treatment. A decrease of 5% can be indicative of effective treatment. A decrease of about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more can indicative of effective treatment. The method can further comprise obtaining a biopsy of the cells labeled by the reagent.

In another aspect, the disclosure provides a method for delivering a therapeutic moiety to a patient comprising the step of administering a reagent provided herein attached to a therapeutic moiety to the patient.

In yet another aspect, the disclosure provides a method for delivering a therapeutic moiety to HCC cells of a patient comprising the step of administering a reagent provided herein attached to a therapeutic moiety to the patient.

In still another aspect, the disclosure provides a kit for administering a composition provided herein to a patient in need thereof, where the kit comprises a composition provided herein, instructions for use of the composition and a device for administering the composition to the patient.

### Linkers, Peptides and Peptide Analogs

As used herein, a “linker” is a sequence of amino acids located at the C-terminus of a peptide of the disclosure. The linker sequence can terminate with, for example, a cysteine or lysine residue.

The presence of a linker can result in at least a 1% increase in detectable binding of a reagent provided herein to HCC cells compared to the detectable binding of the reagent in the absence of the linker. The increase in detectable binding can be at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 100-fold or more.

The term “peptide” refers to molecules of 2 to 50 amino acids, molecules of 3 to 20 amino acids, and those of 6 to 15 amino acids. Peptides and linkers contemplated herein can be 5 amino acids in length. A polypeptide or linker can be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length.

Exemplary peptides are, in various aspects, randomly generated by methods known in the art, carried in a polypeptide library (for example and without limitation, a phage display library), derived by digestion of proteins, or chemically synthesized. Peptides exemplified in the present disclosure have been developed using techniques of phage display, a powerful combinatorial method that uses recombinant DNA technology to generate a complex library of polypeptides for selection by preferential binding to cell surface targets [Scott et al., Science, 249:386-390 (1990)]. The protein coat of bacteriophage, such as the filamentous M13 or icosahedral T7, is genetically engineered to express a very large number (>109) of different polypeptides with unique sequences to achieve affinity binding [Cwirla et al., Proc. Natl. Acad. Sci. USA, 87:6378-6382 (1990)]. Selection is then performed by biopanning the phage library against cultured cells and tissues that over express the target. The DNA sequences of these candidate phage are then recovered and used to synthesize the polypeptide [Pasqualini et al., Nature, 380:364-366 (1996)]. The polypeptides that preferentially bind to FGFR2 are optionally labeled with fluorescence dyes, including but not limited to, FITC, Cy 5.5, Cy 7, and Li-Cor.

Peptides include D and L forms, either purified or in a mixture of the two forms. Also contemplated by the present disclosure are peptides that compete with peptides provided herein for binding to HCC cells.

A peptide of a reagent provided herein can be presented in multimer form. Various scaffolds are known in the art upon which multiple peptides can be presented. A peptide can be presented in multimer form on a trilysine dendritic wedge. A peptide can be presented in dimer form using an aminohexanoic acid linker. Other scaffolds known in the art include, but are not limited to, other dendrimers and polymeric (e.g., PEG) scaffolds.

It will be understood that peptides and linkers provided herein optionally incorporate modifications known in the art and that the location and number of such modifications are varied to achieve an optimal effect in the peptide and/or linker analog.

A peptide analog having a structure based on one of the peptides disclosed herein (the “parent peptide”) can differ from the parent peptide in one or more respects. Accordingly, as appreciated by one of ordinary skill in the art, the teachings regarding the parent peptides provided herein can also be applicable to the peptide analogs.

The peptide analog can comprise the structure of a parent peptide, except that the peptide analog comprises one or more non-peptide bonds in place of peptide bond(s). The peptide analog can comprise in place of a peptide bond, an ester bond, an ether bond, a thioether bond, an amide bond, and the like. The peptide analog can be a depsipeptide comprising an ester linkage in place of a peptide bond.

The peptide analog can comprise the structure of a parent peptide described herein, except that the peptide analog comprises one or more amino acid substitutions, e.g., one or more conservative amino acid substitutions. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The peptide analog can comprise one or more synthetic amino acids, e.g., an amino acid non-native to a mammal. Synthetic amino acids include β-alanine (R-Ala), N-Q-methyl-alanine (Me-Ala), aminobutyric acid (Abu), γ-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (R-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-CI)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4-tetrahydro-isoquinoline-3-carboxylic acid (Tic), tetrahydropyranglycine, thienylalanine (Thi), O-benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid.

The peptide analog can comprise one or more non-conservative amino acid substitutions and the peptide analog still functions to a similar extent, the same extent, or an improved extent as the parent peptide. The peptide analog can comprise one or more non-conservative amino acid substitutions exhibits about the same or greater binding to HCC cells in comparison to the parent peptide.

The peptide analog can comprise one or more amino acid insertions or deletions, in comparison to the parent peptide described herein. The peptide analog can comprise an insertion of one or more amino acids in comparison to the parent peptide. The peptide analog can comprise a deletion of one or more amino acids in comparison to the parent peptide. The peptide analog can comprise an insertion of one or more amino acids at the N- or C-terminus in comparison to the parent peptide. The peptide analog can comprise a deletion of one or more amino acids at the N- or C-terminus in comparison to the parent peptide. In all these instances, the peptide analog still exhibits about the same or greater binding to HCC cells.

### Detectable Markers

As used herein, a “detectable marker” is any label that can be used to identify the binding of a composition of the disclosure to HCC cells. Non-limiting examples of detectable markers are fluorophores, chemical or protein tags that enable the visualization of a polypeptide. Visualization in certain aspects is carried out with the naked eye, or a device (for example and without limitation, an endoscope) and can also involve an alternate light or energy source.

Fluorophores, chemical and protein tags that are contemplated for use herein include, but are not limited to, FITC, Cy5, Cy 5.5, Cy 7, Li-Cor, a radiolabel, biotin, luciferase, 1,8-ANS (1-Anilinonaphthalene-8-sulfonic acid), 1-Anilinonaphthalene-8-sulfonic acid (1,8-ANS), 5-(and-6)-Carboxy-2′, 7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, C5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, eYFP (Enhanced Yellow Fluorescent Protein), FDA, FITC antibody conjugate pH 8.0, FlAsH, Fluo-3, Fluo-3 Ca2⁺, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2⁺, Fura Red, high Ca, Fura Red, low Ca, Fura-2 Ca2+, Fura-2, Fura-2, GFP (S65T), HcRed, Indo-1 Ca2⁺, Indo-1, Ca free, Indo-1, Ca saturated, IDRdye800 (IR800CW), JC-1, JC-1 pH 8.2, Lissamine rhodamine, Lucifer Yellow, CH, Magnesium Green, Magnesium Green Mg2+, Magnesium Orange, Marina Blue, mBanana, mCherry, mHoneydew, mOrange, mPlum, mRFP, mStrawberry, mTangerine, NBD-X, NBD-X, MeOH, NeuroTrace 500/525, green fluorescent Nissl stain-RNA, Nile Blue, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, R-Phycoerythrin pH 7.5, ReAsH, Resorufin, Resorufin pH 9.0, Rhod-2, Rhod-2 Ca2⁺, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodamine Green pH 7.0, Rhodol Green antibody conjugate pH 8.0, Sapphire, SBFI-Na⁺, Sodium Green Na⁺, Sulforhodamine 101, Tetramethylrhodamine antibody conjugate pH 8.0, Tetramethylrhodamine dextran pH 7.0, and Texas Red-X antibody conjugate pH 7.2.

Non-limiting examples of chemical tags contemplated herein include radiolabels. For example and without limitation, radiolabels that contemplated in the compositions and methods of the present disclosure include ¹¹C, ¹³N, ¹⁵O, ¹⁸F, ³²P, ⁵²Fe, ⁶²Cu, ⁶⁴Cu, ⁶⁷Cu, ⁶⁷Ga, ⁶⁸Ga, ⁸⁶Y, ⁸⁹Zr, ⁹⁰Y, ⁹⁴mTc, ⁹⁴Tc, ⁹⁵Tc, ⁹⁹mTc, ¹⁰³Pd, ¹⁰⁵Rh, ¹⁰⁹Pd, ¹¹¹Ag, ¹¹¹n, ¹²³I, ¹²⁴I, ¹²⁵I, ¹³¹I, ¹⁴⁰La, ¹⁴⁹Pm, ¹⁵³Sm, ¹⁵⁴⁻¹⁵⁹Gd, ¹⁶⁵Dy, ¹⁶⁶Dy, ¹⁶⁶Ho, ¹⁶⁹Yb, ¹⁷⁵Yb, ¹⁷⁵Lu, ¹⁷⁷Lu, ¹⁸⁶Re, ¹⁸⁸Re, ¹⁹²Ir, ¹⁹⁸Au, ¹⁹⁹Au, and ²¹²Bi. Gadolinium (Gd) is widely used in complexes and accounts for most of the MR imaging contrast agents applied in the clinic. One example is clinically-approved Gd-GOTA (gadoterate meglumine).

For positron emission tomography (PET) tracers including, but not limited to, carbon-11, nitrogen-13, oxygen-15 and fluorine-18 are used.

A worker of ordinary skill in the art will appreciate that there are many such detectable markers that can be used to visualize a cell, in vitro, in vitro or ex vivo.

### Therapeutic Moieties

Therapeutic moieties contemplated herein include, but are not limited to polypeptides (including protein therapeutics) or peptides, small molecules, chemotherapeutic agents, or combinations thereof.

The term “small molecule”, as used herein, refers to a chemical compound, for instance a peptidometic or oligonucleotide that can optionally be derivatized, or any other low molecular weight organic compound, either natural or synthetic.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

The therapeutic moiety can be a protein therapeutic. Protein therapeutics include, without limitation, cellular or circulating proteins as well as fragments and derivatives thereof. Still other therapeutic moieties include polynucleotides, including without limitation, protein coding polynucleotides, polynucleotides encoding regulatory polynucleotides, and/or polynucleotides which are regulatory in themselves. Optionally, the compositions comprise a combination of the compounds described herein.

Protein therapeutics can include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor β chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein Ill, tumor necrosis factor receptor type I, tumor necrosis factor receptor type II, urokinase-type plasminogen activator receptor, vascular endothelial growth factor, and chimeric proteins and biologically or immunologically active fragments thereof.

Therapeutic moieties can also include chemotherapeutic agents. A chemotherapeutic agent contemplated for use in a reagent provided herein includes, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, capecitabine, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; topoisomerase inhibitors such as irinotecan; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Chemotherapeutic agents such as gefitinib, sorafenib and erlotinib are also specifically contemplated.

Therapeutic moieties to be attached to a peptide described herein also include nanoparticles or micelles that, in turn, encapsulate another therapeutic moiety. The nanoparticles can be polymeric nanoparticles such as described in Zhang et al., ACS NANO, 2(8): 1696-1709 (2008) or Zhong et al., Biomacromolecules, 15:1955-1969 (2014). The micelles can be polymeric micelles such as octadecyl lithocholate micelles described in Khondee et al., J. Controlled Release, 199:114-121 (2015) and WO 2017/096076 (published Jun. 8, 2017). The peptide reagents comprising nanoparticles or micelles can encapsulate, for example, carboplatin, paclitaxel, cisplatin, 5-fluorouracil (5-FU), oxaliplatin, capecitabine or irinotecan.

Dosages of the therapeutic moiety provided are administered as a dose measured in, for example, mg/kg. Contemplated mg/kg doses of the disclosed therapeutics include about 1 mg/kg to about 60 mg/kg. Specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

“Effective amount” as used herein refers to an amount of a reagent provided herein sufficient to visualize the identified disease or condition, or to exhibit a detectable therapeutic or inhibitory effect. The effect is detected by, for example, an improvement in clinical condition or reduction in symptoms. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

### Visualization of Reagents

Visualization of binding to HCC cells is by any means known to those of ordinary skill in the art. As discussed herein, visualization is, for example and without limitation, in vitro, in vitro, or in situ visualization.

When the detectable label is a radiolabel, the radiolabel can be detected by nuclear imaging.

When the detectable label is a fluorophore, the fluorophore can be detected by near infared (NIR) fluorescence imaging.

When the detectable label has magnetic properties, it can be detected by magnetic resonance (MR) imaging.

Methods provided herein can comprise the acquisition of a tissue sample from a patient. The tissue sample can be a tissue or organ of said patient.

### Formulations

Compositions provided herein are formulated with pharmaceutically acceptable excipients such as carriers, solvents, stabilizers, adjuvants, diluents, etc., depending upon the particular mode of administration and dosage form. The compositions are generally formulated to achieve a physiologically compatible pH, and range from a pH of about 3 to a pH of about 11, about pH 3 to about pH 7, depending on the formulation and route of administration. The pH can be adjusted to a range from about pH 5.0 to about pH 8. The compositions can comprise a therapeutically effective amount of at least one reagent as described herein, together with one or more pharmaceutically acceptable excipients. Optionally, the compositions comprises a combination of the compounds described herein, or can include a second active ingredient useful in the treatment or prevention of bacterial growth (for example and without limitation, anti-bacterial or anti-microbial agents), or can include a combination of reagents provided herein.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol) wetting or emulsifying agents, pH buffering substances, and the like.

As used herein, “can comprise” or “can be” indicates something contemplated by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

### Example 1

### Generation and Characterization of a Peptide Specific for CD44

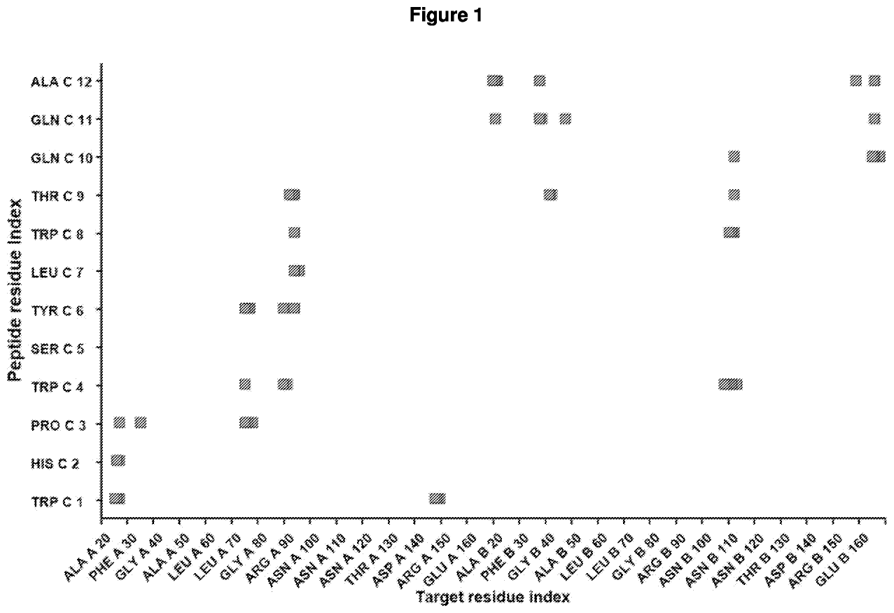

A library of candidate peptide sequences was formed by analyzing a contact map (FIG. 1) for binding activity to the extracellular hyaluronan binding domain of CD44 (1 UUH).

The crystal structure (1 UUH) for the extracellular hyaluronan binding domain of CD44 was obtained from the Protein Data Bank (PDB) [Juliano et al., Wiley Interdiscip Rev Nanomed Nanobiotechnol 2009, 1, 324-335]. CABS-dock software [Ji, supra; Lee et al., Chem Rev 2010; 110:3087-111] was used to explore possible peptide binding sites to this CD44 domain and to evaluate alignment [Zhang et al., Chem Soc Rev 2018; 47:3490-3529]. This software enables full flexibility of the peptide structure and large-scale flexibility of protein fragments during an empiric search for binding sites. Pairs of peptide/target residues with inter molecular distance<4.5 Å were selected to optimize binding affinity and specificity. The peptide pairs are shown in Table 1.

TABLE 1

| Receptor residue | Peptide residue |
|---|---|
| ASP B 167 | ALA C 12 |
| GLU B 166 | GLN C 10 |
| GLN B 113 | GLN C 10 |
| SER B 112 | TRP C 8 |
| THR B 111 | TRP C 4 |
| GLU B 48 | GLN C 11 |
| ASN B 39 | GLN C 11 |
| ILE B 22 | ALA C 12 |
| ALA B 20 | ALA C 12 |
| VAL A 148 | TRP C 1 |
| ASN A 94 | TRP C 8 |
| PRO A 93 | THR C 9 |
| ARG A 90 | TYR C 6 |
| CYS A 77 | TYR C 6 |
| GLU A 75 | TYR C 6 |
| HIS A 35 | PRO C 3 |
| THR A 27 | TRP C 1 |
| ASN A 25 | TRP C 1 |
| TYR B 169 | GLN C 10 |
| ASP B 167 | GLN C 10 |
| TYR B 114 | TRP C 4 |
| GLN B 113 | TRP C 8 |
| THR B 111 | TRP C 8 |
| SER B 109 | TRP C 4 |
| TYR B 42 | THR C 9 |
| LYS B 38 | GLN C 11 |
| GLN B 21 | GLN C 11 |
| ASN A 149 | TRP C 1 |
| ASN A 94 | THR C 9 |
| ASN A 94 | TYR C 6 |
| ILE A 91 | TRP C 4 |
| ARG A 78 | PRO C 3 |
| THR A 76 | PRO C 3 |
| GLU A 75 | PRO C 3 |
| THR A 27 | HIS C 2 |
| ILE A 26 | TRP C 1 |
| ASP B 167 | GLN C 11 |
| GLU B 160 | ALA C 12 |
| GLN B 113 | THR C 9 |
| SER B 112 | TRP C 4 |
| ASN B 110 | TRP C 4 |
| SER B 43 | THR C 9 |
| LYS B 38 | ALA C 12 |
| GLN B 21 | ALA C 12 |
| ARG A 150 | TRP C 1 |
| ILE A 96 | LEU C 7 |
| ASN A 94 | LEU C 7 |
| HIS A 92 | THR C 9 |
| ARG A 90 | TRP C 4 |
| THR A 76 | TYR C 6 |
| GLU A 75 | TRP C 4 |
| THR A 27 | PRO C 3 |
| ILE A 26 | HIS C 2 |

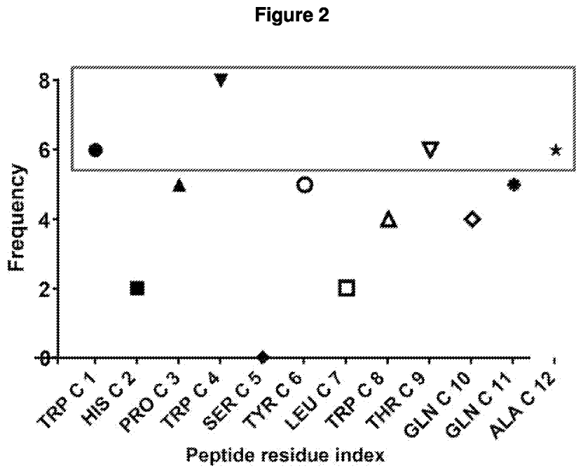

Amino acids that paired more than five times were recognized as having high correlation and affinity, and were preserved in the peptide sequence being designed (FIG. 2, red box). The general peptide sequence WX1X2WX3X4X5X6TX7X8A was used where X1 represents either H or K to form hydrophilic interactions with CD44 at the N-terminus. X2 represents either P or G which often form “turns” of peptides. For sites X3-X6, amino acids with different properties were chosen to increase sequence diversity. X3 represents S or N; X4 represents Y, A, I or F; X5 represents L, A, I or F; X6 represents W, A, I or F. X7-X8 at the C-terminus represents negative charged Q or D which have an electrostatic repulsion to the negative extraneous coat of cells to reduce peptide entry into the cells. In the library of peptides generated, from X1-X8, the sequence was randomly distributed, resulting in a complexity of 2×2×2×4×4×4×2×2=2048. Hex 8.0.0 protein-ligand docking software was then used to evaluate binding of each candidate peptide to the CD44 hyaluronan binding domain [Feng et al., J Med Chem 2021 Sep. 30. doi: 10.1021/acs.jmedchem.1c00697]. This program comprehensively evaluates all possible combinations for the predicted binding motifs of each candidate sequence, and calculates the docking energy for binding between the peptide and target. Hex 8.0.0 was also used to identify a scrambled sequence for use as control.

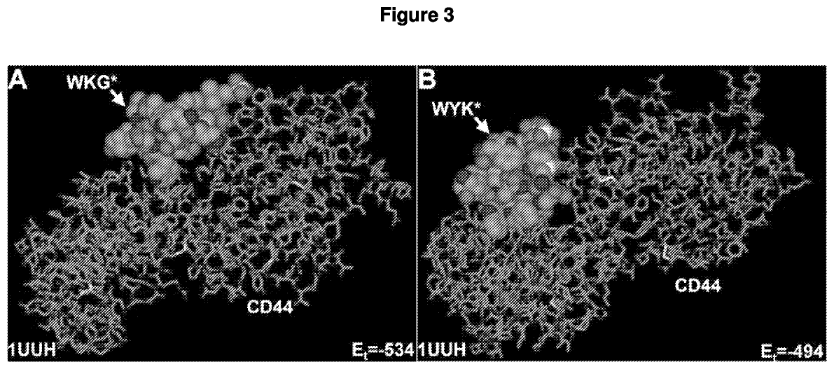

The peptide with a sequence WKGWSYLWTQQA (SEQ ID NO: 1), hereafter WKG*, was found to bind CD44 with a total energy Eₜ=−534 (FIG. 3A) and was chosen for further development. This sequence was scrambled to generate a peptide WYKAQQWWTLGS (SEQ ID NO: 3), hereafter WYK*, for use as control and resulted in Eₜ=−494, FIG. 3B).

### Peptide Synthesis

The target and control peptides were synthesized using standard Fmoc-mediated solid-phase chemical synthesis on rink amide MBHA resin using a PS3 automatic synthesizer (Protein Technologies Inc). Fmoc (Fluorenylmethyloxycarbonyl) and Boc (Butyloxycarbonyl) protected L-amino acids were used with standard HBTU/HOBt activation. After assembly, the resin was washed with dimethylformamide (DMF) and dichloromethane (DCM), cleaved with a trifluoroacetic acid cocktail (TFA: thioanisole: phenol: EDT: H2O, 87.5:5:2.5:2.5:2.5, v/v/v/v/v). The resulting peptide was precipitated in −20° C. diethyl ether. The crude peptides were then purified using reversed-phase high performance liquid chromatography (RP-HPLC). The purified peptide was lyophilized to produce a white powder, and was characterized with MALDI-TOF mass spectrometry.

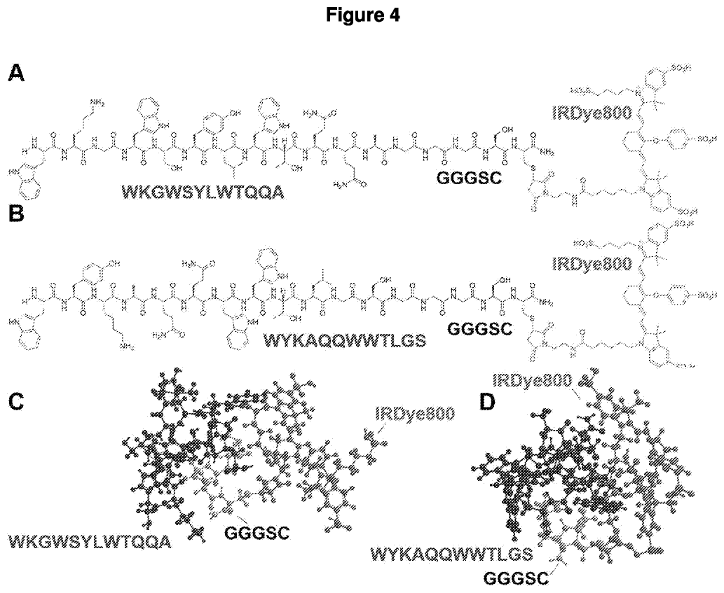

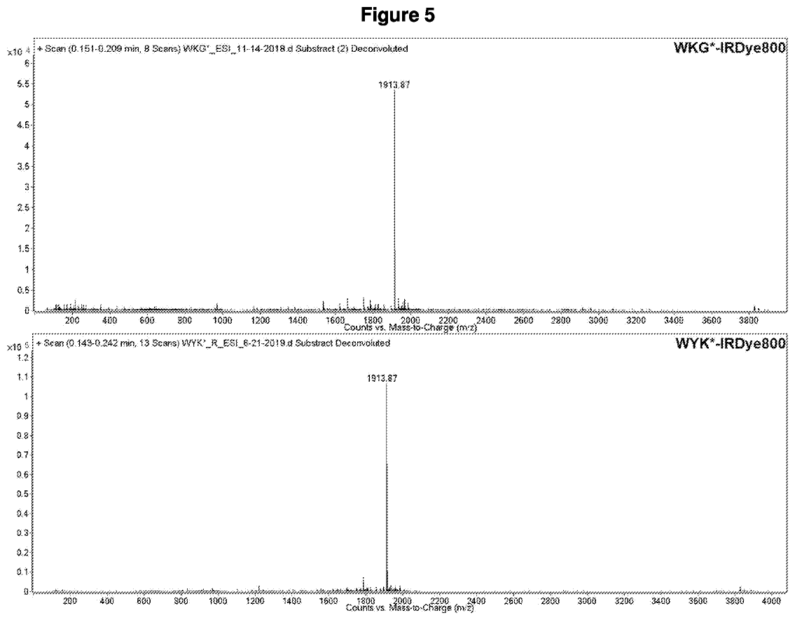

The C-terminus of the CD44-directed peptide was covalently linked with IRDye800, a near-infrared (NIR) fluorophore, via a GGGSC linker, hereafter WKG*-IRDye800, FIG. 4A. The linker separates the peptide from the fluorophore and prevents steric hindrance. The scrambled sequence was also labeled with IRDye800, hereafter WYK*-IRDye800, FIG. 4B. 3D models are shown to highlight differences between the biochemical structures, FIG. 4C,D. The peptides were synthesized with >95% purity by HPLC, and an experimental mass-to-charge ratio (m/z) of 1913.87 was measured using mass spectrometry, which agrees with expected value of 1913.88, FIG. 5A,B.

### Spectral Measurements

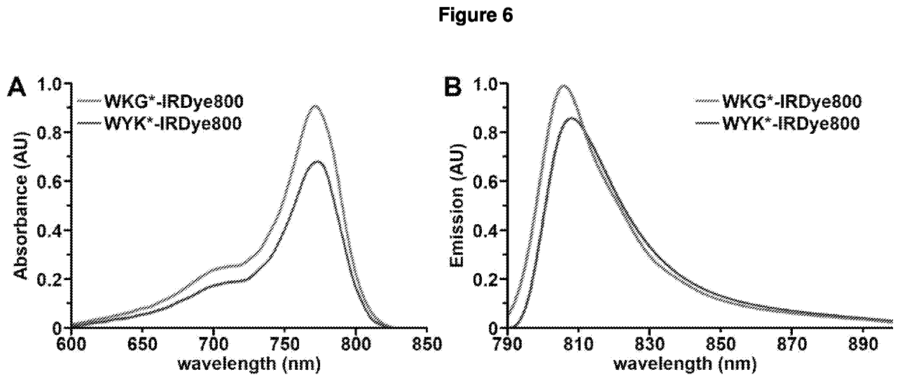

The absorbance spectrum of the peptides was measured using a UV-Vis spectrophotometer (NanoDrop 2000c, Thermo Scientific). The peptides were excited at λₑₓ=785 nm with a single-mode diode laser (#iBEAM-SMART-785-S, TOPTICA Photonics), and FL emission was collected using a spectrometer (USB2000+, Ocean Insight). The spectra were plotted using Prism 5.0 software (GraphPad Inc). Peak absorbance and emission occur in the near-infrared (NIR) spectrum where hemoglobin absorption, tissue scattering, and tissue autofluorescence are minimal, FIG. 6A,B. siRNA knockdown

CD44 expression in SK-Hep1 cells was knocked down using three different siRNAs, including 1) L-009999-00-0005, Dharmacon; 2) s2681, Thermo Fisher; and 3) 106160, Thermo Fisher. MISSION® siRNA Universal Negative Control (SIC001, Sigma) was used for control. Cells were transfected with Lipofectamine 2000 (11668027, Invitrogen) per manufacturer instructions, and then incubated with 4 μM of peptide for 3 min. A 1:3000 dilution of rabbit anti-CD44 antibody (EPR18668, Abcam) was used for positive control. CD44 expression was determined by Western blot within 72 hours.

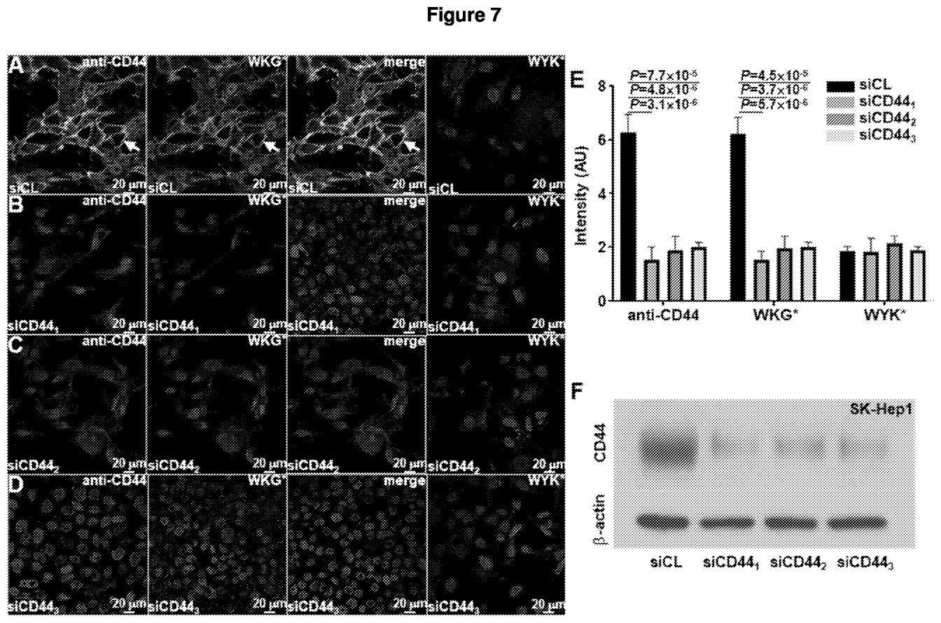

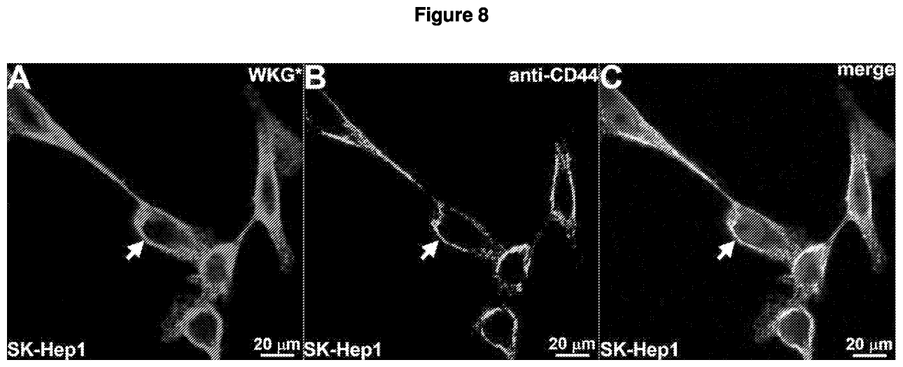

CD44 expression was knocked down in human SK-Hep1 HCC cells using siRNA to validate specific binding of WKG*-IRDye800 to CD44. WKG*-IRDye800 and anti-CD44-AF488 antibody showed strong binding to the surface (arrows) of SK-Hep1 cells transfected with siCL (control) using confocal microscopy, while WYK*-IRDye800 displayed minimal binding, FIG. 7A. Fluorescence intensities from SK-Hep1 cells with knockdown of CD44 showed minimal intensity with either peptide, FIG. 7B-D. Quantified results showed this decrease to be significant, FIG. 7E. Western blot confirms effective knockdown of CD44 in SK-Hep1 cells, FIG. 7F. Binding by WKG*-IRDye800 and anti-CD44-AF488 to the surface (arrows) of Sk-Hep1 cells co-localizes with a correlation of p=0.81 measured on the merged image, FIG. 8.

### Confocal Fluorescence Microscopy

Approximately 10³ SK-Hep1 and Hep 3B cells were grown on cover glass in 24-well plate to ˜70% confluence. The cells were washed with PBS 1× and incubated with 4 μM of either target or control peptide for 3 min. The cells were then washed 3× in PBS, fixed with 4% paraformaldehyde (PFA) for 8 min, washed 3× with PBS then incubated with 2% BSA, 1% goat serum in PBS for 30 min. The cells were incubated with a 1:3000 dilution of primary recombinant rabbit anti-CD44 antibody (#ab189524, Abcam) for 30 min on ice and then incubated with a 1:500 dilution of AF488-labeled secondary goat ant-rabbit immunoglobulin G antibody (#A-11029, Life Technologies) for 12 hours at 4° C., and then mounted on glass slides with ProLong Gold reagent containing DAPI (Invitrogen). Confocal fluorescence images were collected on Leica SP8 confocal microscope using a 63× oil-immersion objective. Fluorescence intensities were quantified using custom MATLAB (Mathworks) software.

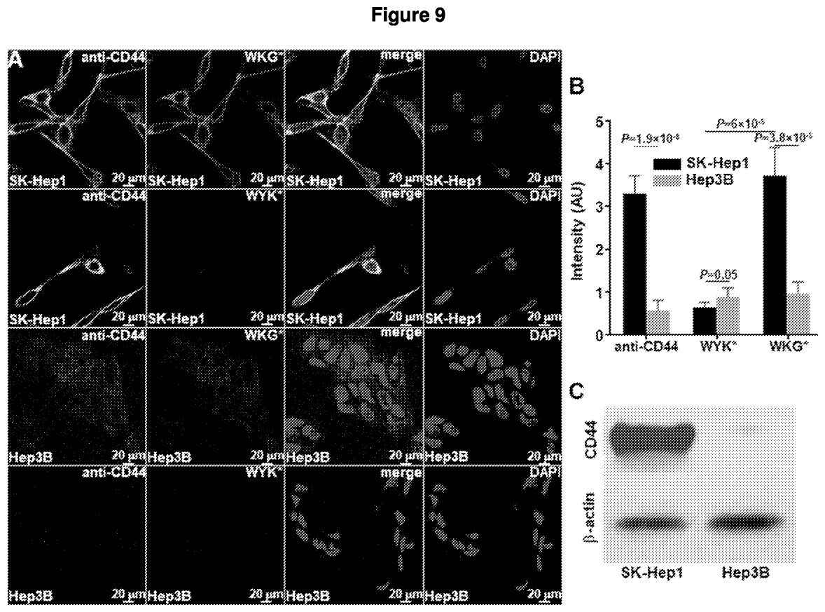

Significantly greater fluorescence intensity was observed for binding of WKG*-IRDye800 and anti-CD44-AF488 to SK-Hep1 cells (CD44+) compared with Hep 3B cells (CD44⁻) cells, FIG. 9.

### Example 2

### Further Peptide Characterization

Specific peptide binding to CD44 was further validated using competitive inhibition by adding unlabeled peptide. Approximately 103 SK-Hep1 cells were grown to ˜70% confluence on cover glass in triplicate. Unlabeled peptides at concentrations of 0, 10, 20, 40, 80, and 100 μM were incubated with the cells for 30 min at 4° C. The cells were washed with PBS and incubated with 5 μM of the target peptides for another 30 min at 4° C. The cells were washed and fixed with 4% PFA for 8 min. The cells were washed with PBS and mounted with ProLong Gold reagent containing DAPI (Invitrogen).

The apparent dissociation constant k_{d} for peptide binding to cells was measured to assess the binding affinity [31]. IRDye800-labeled target peptides were serially diluted in PBS at concentrations of 0, 10, 20, 40, 80, 100, and 200 nM. ˜10⁵ SK-Hep1 cells were incubated with the peptides at 4° C. for 1 hour, washed with cold PBS, and the mean fluorescence intensities were measured using flow cytometry. The equilibrium dissociation constant k_{d}=1/kₐ was calculated by performing a least squares fit of the data to the non-linear equation l=(l₀+lₘₐₓkₐ [X])/(l₀+kₐ[X]). l₀ and lₘₐₓ are the initial and maximum fluorescence intensities, corresponding to no peptide and at saturation, respectively, and [X] represents the concentration of the bound peptide. Prism 5.0 software (GraphPad Inc) was used to calculate k_{d}.

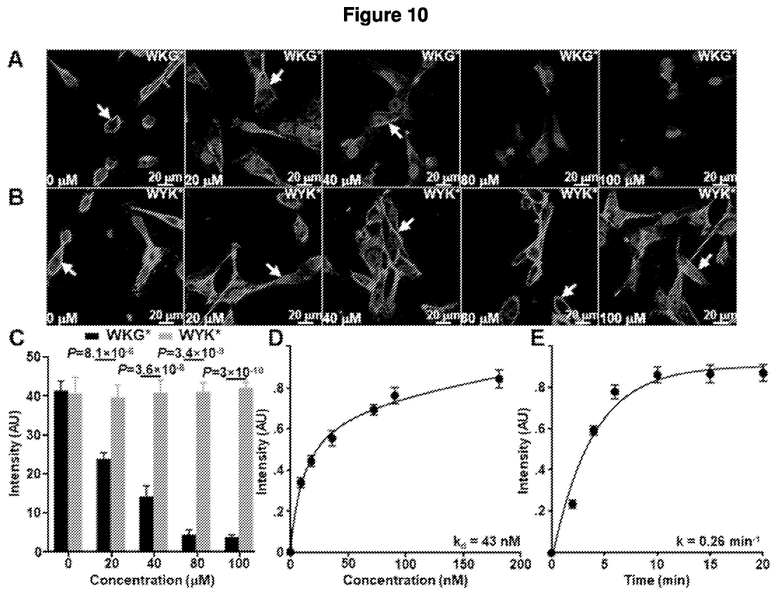

Specific binding of WKG*-IRDye800 to CD44 was further supported by addition of unlabeled WKG* to compete for binding. Fluorescence intensities from SK-Hep1 cells decreased significantly with increasing concentrations of unlabeled WKG*, FIG. 10A, but not with WYK*, FIG. 10B. Quantified results show the decrease to be concentration dependent, FIG. 10C. These results suggest that the peptide rather than either the linker or fluorophore mediates the binding interactions. An apparent dissociation constant of k_{d}=43 nM was measured for binding by WKG*-IRDye800 to SK-Hep1 cells using flow cytometry, FIG. 10D. An apparent association time constant of k=0.26 min⁻¹ (6.8 min) was measured to support rapid binding onset, FIG. 10E.

### Example 3

### Effect of Peptide on Cell Signaling

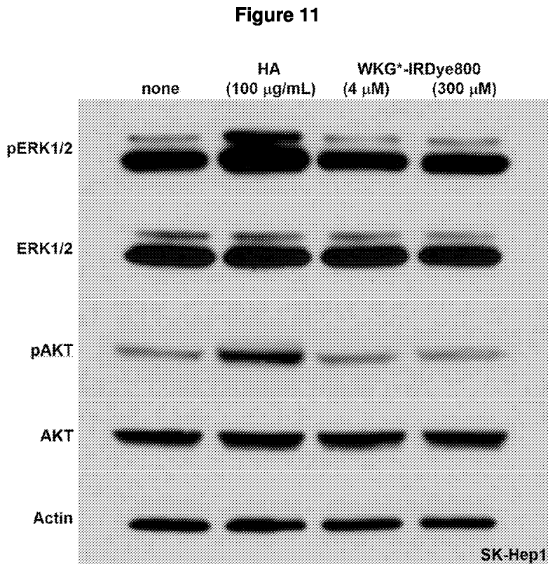

Western blots were performed to evaluate markers for activation of downstream cell signaling, FIG. 11. SK-Hep1 cells were incubated with either hyaluronan (HA) or peptide to evaluate activation of downstream signalling after binding to CD44. 100 ag/mL of low molecular weight HA (GLR001, R&D Systems) was added for 15 min. Peptides are added at concentrations of 4 and 300 μM for 15 min. Anti-CD44 antibody (#ab189524, Abcam), anti-AKT (#4691, Cell Signaling), anti-phospho-AKT (#9271, Cell Signaling), anti-ERK1/2 (#ab17942, Abcam,), anti-phospho-ERK1/2 (#ab50011, Abcam), and anti-β-Actin (#4967, Cell Signaling Technology) were used per manufacturer's instructions.

Incubation of low molecular weight hyaluronan (HA) as a positive control with SK-Hep1 cells showed strong phosphorylation activity for downstream AKT and ERK1/2, including pAKT and pERK1/2, respectively. By comparison, addition of WKG*-IRDye800 at various concentrations resulted in no change in phosphorylation of downstream substrates.

### Example 4

### Test for Cytotoxicity

The CD44-binding and control peptides were serially diluted over a range of concentrations and incubated with SK-Hep1 cells seeded in 96-well plates for 24 hours. The media was then removed, and MTT solution (100 μL, 0.5 mg/mL) was added. After 4 hours for incubation, the MTT solution was removed, and 150 μL of DMSO was added to each well to solubilize formazan crystals produced by living cells. Absorbance at A_{abs}=570 (test) and 630 nm (reference) was measured using a plate reader (VersaMax™ Tunable Microplate Reader). The half-maximum inhibitory concentration (IC₅₀) was measured.

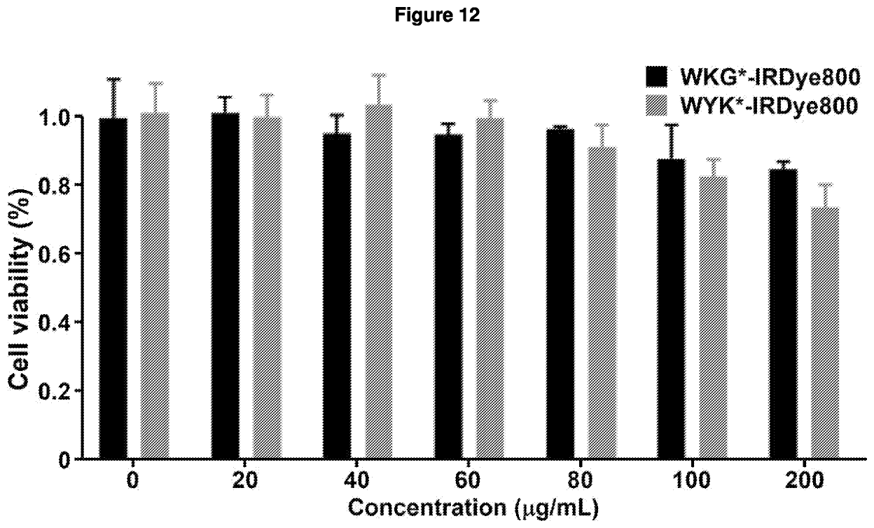

An MTT assay was performed to evaluate the cytotoxicity of CD44 peptides. Peptides were incubated with SK-Hep1 cells in increasing concentrations up to 200 μg/mL for 24 hours. The WKG*-IRDye800 and WYK*-IRDye800 peptides showed no effect on cell viability until the highest concentrations were reached, FIG. 12.

### Example 5

### Serum Stability

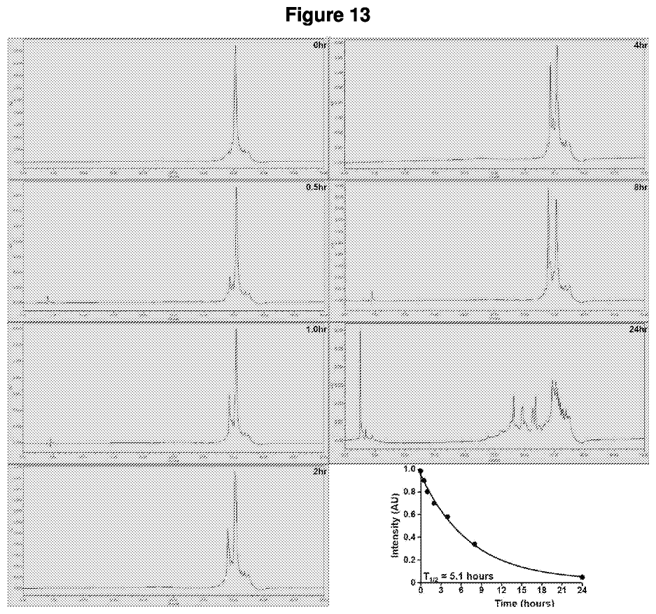

To evaluate the serum stability of WKG*-IRDye800, the peptide was incubated with mouse serum up to 24 hours, and then measured by analytical RP-HPLC, FIG. 13. The relative concentration was determined by the area-under-the-peak (Breeze 2, Waters), and a half-life of T_{1/2}=5.1 hours was measured, R²=0.99, FIG. 13.

### Example 6

### In Vitro Photoacoustic Imaging of Orthotopic Human HCC Xenograft Tumors

Human HCC xenograft tumors were implanted orthotopically in female nude athymic mice. First, ˜5×10⁶ SK-Hep1 tumor cells were injected subcutaneously into the hind limb flank. Tumors were then monitored twice a week and allowed to grow to 1-2 cm in diameter for 10-30 days. A small horizontal incision was made below the sternum to expose the liver. The liver was incised with a sharp scalpel horizontally in parallel with the surface of the exposed liver. A piece of the subcutaneous tumor with dimensions of ˜1×1×1 mm³ was implanted into the incision, and then the site was sealed with absorbable hemostatic material (surgical, Johnson & Johnson). The liver was returned to its original position after confirming hemostasis.

IRDye800-labeled target and control peptides (300 μM in 200 μL PBS) were intravenously injected in mice bearing orthotopical SK-Hep1 tumors. An unlabeled peptide (1.5 mM, 100 μL) was injected 30 min prior to the labeled peptide to compete for binding. ICG (2.46 mg/kg) was injected intravenously as a control. Three-dimensional (3D) images were acquired from 0 to 48 h post injection and reconstructed using PAI tomography system (Nexus 128, Endra) using Iₑₓ=774 nm excitation. The photoacoustic signal intensity was measured from the two-dimensional (2D) maximum intensity projection (MIP) images, and the pre-injection images were used for background.

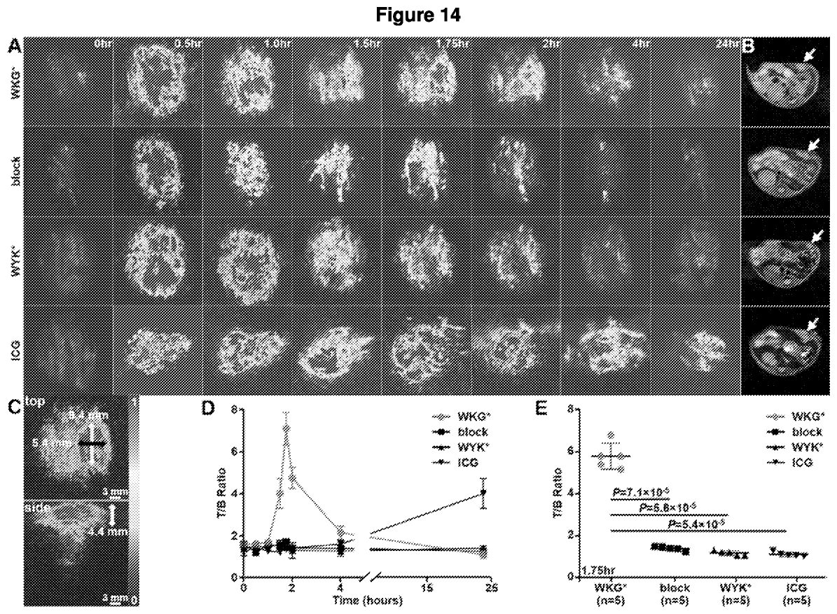

The photoacoustic images were collected to evaluate the time course for peptide uptake, FIG. 14A. Minimal intensity was observed from the tumors prior to peptide injection (0 hr). Following intravenous administration of WKG*-IRDye800, the intensity peaked at 1.75 hours post-injection, and returned to baseline by ˜24 hours. Unlabeled WKG* (7 mM, 200 μL) was injected 20 min prior to WKG*-IRDye800 to compete for binding to CD44. Decreased signal was observed from the tumors over time. WYK*-IRDye800 was administered systemically for control, and showed reduced intensity. Indocyanine green (ICG) was also administered (2.46 mg/kg) for comparison. Peak uptake of ICG was not observed within 24 hours post-injection. T-weighted MR images were collected from tumor-bearing mice to confirm the presence of orthotopically implanted HCC tumor (arrow), FIG. 14B. A 3D reconstruction shows the tumor dimensions, FIG. 14C. Quantified intensities confirms peak uptake of WYK*-IRDye800 in tumor at 1.75 hours post-injection, and returned to baseline by ˜24 hours FIG. 14E. The mean T/B ratio for WKG* was found to be significantly greater than that for block, WYK*, and ICG at peak uptake, FIG. 14E.

### Example 7

### In Vitro Whole Body Imaging of Orthotopic Human HCC Xenograft Tumors

SK-Hep1 tumor bearing mice (generated as described in Example 6) were injected intravenously with the IRDye800-labeled target and control peptides (300 μM in 200 μL PBS). The spatial extent and margins of tumors were identified using a NIR whole body fluorescence imaging system (Pearl®, LI-COR Biosciences) up to 24 h post injections. The images were acquired using λₑₓ=800 nm with 85 μm resolution and 16.8×12 cm² field of view (FOV). Image Studio software (Li-Cor Biosciences) was used for analysis. Regions of interest (ROI) with area equal to that of the tumor and adjacent in location was measured for background.

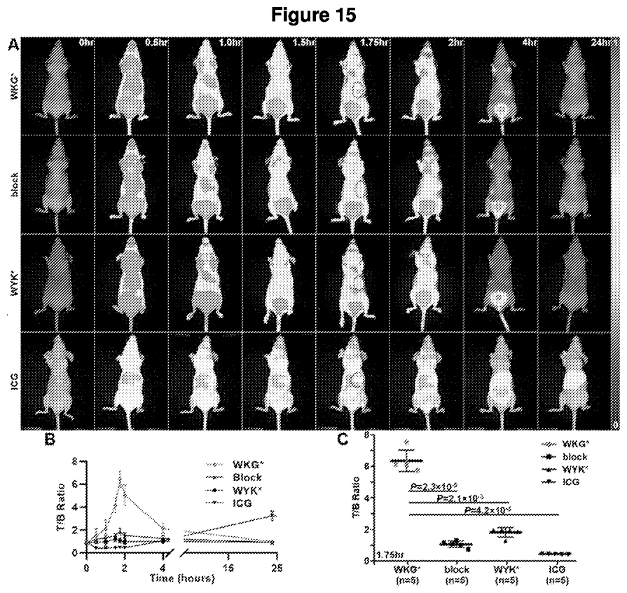

Whole body fluorescence images collected from orthotopic SK-Hep1 xenograft tumors showed minimal intensity prior to peptide injection (0 hr), FIG. 15A. Following intravenous administration of WKG*-IRDye800, the intensity peaked at 1.75 hours post-injection, and returned to baseline by ˜24 hours. Unlabeled WKG* (7 mM, 200 μL) was injected 20 min prior to WKG*-IRDye800 to compete for binding to CD44 (block), and decreased fluorescence intensities were observed from the tumors at each time point. WYK*-IRDye800 was administered systemically for control, and showed reduced intensity. ICG was also administered as a comparison, and showed strong background up to 24 hours. Quantified intensities confirmed peak uptake of WYK*-IRDye800 in tumor at 1.75 hours post-injection, and returned to baseline by ˜24 hours FIG. 15B. The mean T/B ratio for WKG* was found to be significantly greater than that for block, WYK*, and ICG at peak uptake, FIG. 15C.

### Example 8

### Intraoperative Laparoscopic Imaging of Orthotopic Human HCC Xenograft Tumors

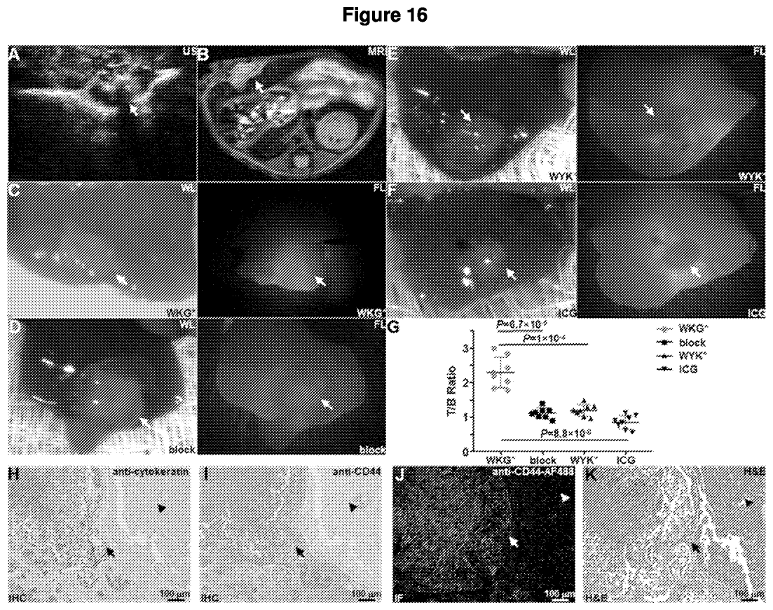

Ultrasound (US) and T₁-weighted MR images were collected from mice (generated as described in Example 6) to confirm the orthotopic location of implanted HCC tumors (arrow), FIG. 16A,B.

A self-build imaging module was attached to standard surgical laparoscope (#49003 AA, HOPKINS II Straight Forward Telescope 0°, Karl Storz, El Segundo, CA, USA) to collect WL and NIR FL images. WL illumination (MCWHL5, Thorlabs, Newton, NJ, USA) and FL excitation source (λₑₓ=785 nm, #iBEAM-SMART-785-S, Toptica Photonics) were coupled into the laparoscope. WL and NIR FL images are collected, simultaneously, by a color CCD camera (#GX-FW-28S5C-C, Point Grey Research, Richmond, BC V6W 1 K7, Canada) and a NIR CCD camera (Orca R-2, Hamamatsu Photonics, Hamamatsu City, Shizuoka Pref., Japan) with a laser power of 1.2 mW, respectively.

WKG*-IRDye800, unlabeled WKG* (block), WYK*-IRDye800, and ICG were administered systemically 1.75 hours prior to imaging. Representative white light and fluorescence images collected in vitro from the exposed liver are shown, FIG. 16C-F. Image intensities were quantified, and the mean T/B ratio for WKG* was significantly greater than that for block, WYK*, and ICG, FIG. 16G. After completion of imaging, the mice were euthanized, and the livers were resected. A human specific anti-cytokeratin was stained on tumor sections by IHC to further confirm the implanted human derived HCC tumors, FIG. 16H. Overexpression of CD44 was confirmed by IHC and IF, FIG. 161,J. Representative histology (H&E) of the tumor is shown, FIG. 16K.

### Example 9

### Peptide Biodistribution

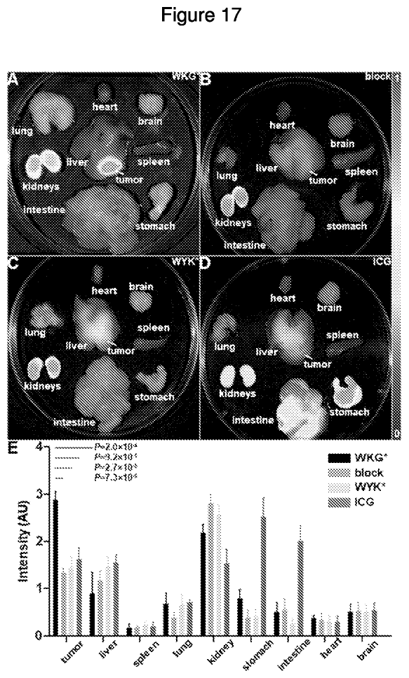

Tumor-bearing mice generated as described in Example 6 were sacrificed at 1.75 hours post-injection of WKG*-IRDye800, WYK*-IRDye800, WKG*, and ICG. The animals were euthanized at peak uptake after intravenous injection of the target and control peptides. Major organs, including heart, spleen, lung, liver, brain, stomach, kidney, intestine, were resected and exposed for white light and fluorescence imaging to measure peptide biodistribution. White light and NIR fluorescence images were collected from the major organs, FIG. 17.

Uptake of WKG*-IRDye800 in the tumor was found to be significantly higher versus that of other groups. For WYK* and WKG*, low uptake was observed in all other organs except the kidneys where the peptide is cleared. ICG showed strong signal from stomach and intestine due to the different route of body clearance.

### Example 10

### Animal Necropsy

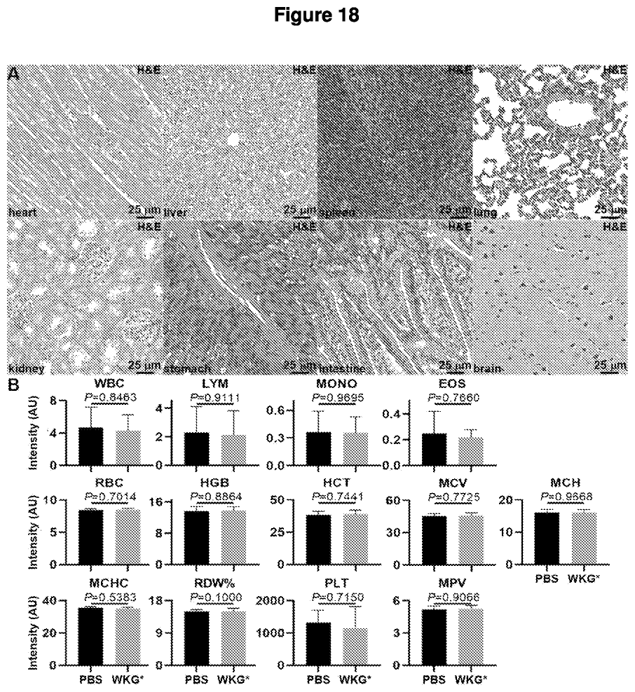

After systemic administration of WKG*-IRDye800 for 48 hours, normal healthy mice were euthanized. Whole blood was collected for evaluation of hematology and chemistry. Liver, kidney, heart, lung, spleen, stomach, intestine, and brain were harvested and submitted for routine histology (H&E). All slides were evaluated by a liver pathologist. No signs of toxicity were seen in heart, liver, spleen, lung, kidney, stomach, intestine and brain, FIG. 18A. No acute peptide toxicity was observed, FIG. 18B.

### Example 11

### Peptide Validation in Human HCC Specimens Ex Vivo

A tissue microarray (TMA) of human HCC was generated to investigate specific binding by the CD44 peptide to human HCC. Formalin-fixed, paraffin-embedded (FFPE) sections of human liver were obtained from the archived tissue bank in the Department of Pathology. The specimens were washed 3× in xylene for 3 min, 100% ethanol for 3 min, 95% ethanol for 3 min, 70% ethanol for 3 min, rinsed in H₂O for 2 min. Antigen unmasking was performed by boiling the slides in 10 mM sodium citrate buffer with 0.05% Tween at pH 6.0 for 15 min. The slides were cooled to RT, and were washed in H₂O 3× for 5 min. Blocking was performed with DAKO protein blocking agent (X0909, DAKO) for 1 hour at RT. The peptides at 1 μM concentration were incubated for 10 min at RT. The sections were washed 3× in PBST for 3 min, and incubated with 400 μL at 1:500 dilution of recombinant anti-CD44 (#ab189524, Abcam) overnight at 4° C. The sections were then washed 3× in PBST for 5 min. A 1:500 dilution of AF488-labeled secondary antibody (goat anti rabbit Alexa Fluor®488) was added to each section and incubated for 1 hour at RT. The secondary antibody solution was removed and washed 3× with PBST for 5 min. The sections were then mounted with ProLong Gold reagent containing DAPI (Invitrogen). The fluorescence images of each specimen were collected using confocal microscopy (SP8, Leica), and the mean fluorescence intensity from each image was measured from 3 boxes with dimensions of 20×20 μm² using custom MATLAB software. Regions of saturated image intensities were avoided.

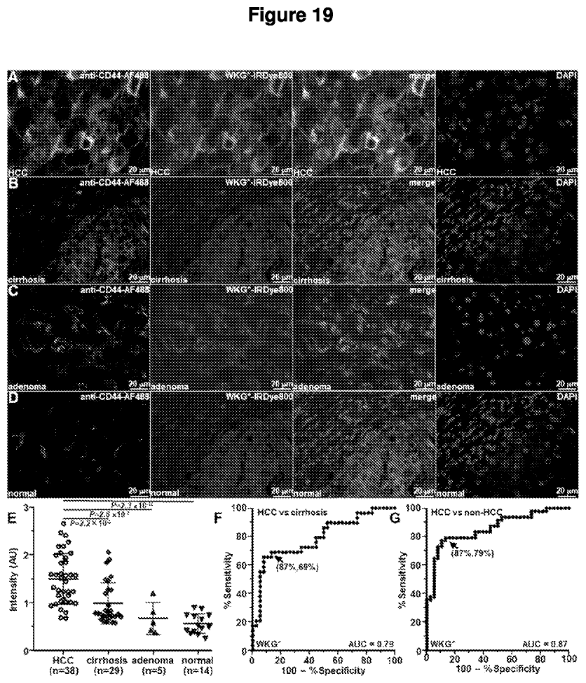

Both peptide and antibody showed intense staining for HCC, FIG. 19A. Minimal staining to adenoma and moderate diffuse staining to cirrhosis were observed, FIG. 19B,C. A representative section of normal human liver showed negligible staining, FIG. 19D. Results were compared with histology interpreted by an expert liver pathologist (EYC). The fluorescence intensities were quantified, and the mean (±SD) values were significantly greater for HCC than for the other histological classifications, FIG. 19E. The ROC curve shows 87% sensitivity and 69% specificity for distinguishing HCC from cirrhosis with AUC=0.79, FIG. 19F, and 87% sensitivity and 79% specificity for distinguishing HCC from all non-HCC with AUC=0.87, FIG. 19G.

### Summary of Examples 1-11

Two-sided Welch's two-sample t-tests were performed to assess the specific binding of WKG* to HCC cells, which allow unequal variance in the two groups being compared. All tests were performed at the Bonferroni-corrected significance level α=0.05/m, where m is the total number of statistical tests performed, to account for the multiple comparisons made between WKG* and various controls. For example, if three controls are present, each individual test would be performed at α=0.05/3=0.017, and if three controls were examined in nine tissues, each target peptide versus control test would be performed at α=0.05/27=0.0019.

As described above, a structural model was used to optimize the sequence of a 12-mer peptide (WKG*) for specific binding to CD44. The peptide was labeled with IRDye800, and specific binding was validated in vitro with knockdown, competition, and co-localization studies. Binding properties of the labeled peptide WKG*-IRDye800 were characterized by an apparent dissociation constant of k_{d}=43 nM and apparent association time constant of k=0.26 min⁻¹ (6.8 min). Human HCC cells were implanted orthotopically in mouse liver, and peak uptake by tumor in vitro was observed at 1.75 hours post-injection using photoacoustic imaging. Specific WKG*-IRDye800 peptide uptake was supported by fluorescence images collected using whole body and laparoscopic imaging. Specific WKG*-IRDye800 peptide binding to CD44 in vitro was further confirmed by blocking the targeted contrast agent with unlabeled peptide. Ex vivo staining results using human HCC specimens support the ability of the WKG*-IRDye800 peptide to distinguish HCC from other liver pathologies. No evidence of toxicity was observed on animal necropsy.

Previous peptides specific for CD44 have been reported. A peptide was selected using biopanning with a M13 phage display library for detection of breast cancer [Park et al., Mol Biotechnol 51(3) (2012) 212-20.]. A binding affinity of 115.8 and 256.5 nM was measured for FITC-labeled and biotinylated peptides, respectively. No in vivo imaging was performed. A peptide specific for CD44 was developed for early detection of gastric cancer [Zhang et al, World J Gastroenterol. 2012; 18:2053-60; Zhang et al., Biotechnol Lett 2015; 37:2311-20; Zhang et al., J Biomol Screen 2016; 21:44-53; Li et al., Oncotarget 2017; 8:30063-30076]. Docking to CD44 was evaluated using a structural model, and a binding affinity of k_{d}=135.1 nM was reported. Fluorescence imaging was performed in subcutaneous gastric tumors, and a peak T/B ratio in tumor was detected three hours post-injection. The biodistribution showed accumulation in both tumor and liver. Also, a peptide specific for CD44v6 was identified, and a binding affinity of k_{d}=611.2 nM was reported [Zhang et al., Ann Transl Med 2020; 8(21):1442.]. By comparison, the peptide WKG*-IRDye800 herein showed 3-fold improved binding affinity, and demonstrated primarily renal clearance. This pathway is preferred because accumulation of the contrast agent in the liver can increase background to limit imaging performance.

Multi-modal imaging methods were used to rigorously validate specific WKG*-IRDye800 peptide binding to CD44 in vitro. First, ultrasound and MRI were used to confirm the orthotopic location of HCC tumors. Photoacoustic and fluorescence imaging methods provide different physical mechanisms by which signal is generated from the NIR-labeled peptide to confirm specific ligand binding to the CD44 target. The photoacoustic images combined light with sound to visualize the depth of peptide accumulation in tumor. The whole body fluorescence images demonstrated the spatial distribution of peptide uptake for comparison of tumor with the other body organs. Both modalities showed peak tumor uptake at 1.75 hour post-injection, and clearance by −24 hour. The WKG*-IRDye800 peptide was found to be stable in serum for over 5 hours. Fluorescence laparoscopy was performed intraoperatively, and demonstrated sharp tumor margins within normal mouse liver parenchyma. Ultrasound and MRI were used to confirm the orthotopic location of the HCC tumors. These results are compatible with future clinical use as a diagnostic imaging agent for early detection of HCC and for image-guided surgery.

Image-guided surgery is being used with greater frequency to more precisely resect HCC tumors. The intraoperative diagnosis of small tumors, especially those with indistinct margins, remains an important challenge for HCC resection. Thus, a specific targeting agent has potential to significantly improve diagnostic performance during laparoscopy. Experienced surgeons can achieve very good patient outcomes with 5-year survival rates over 70% for solitary early-stage HCC. ICG is FDA-approved, and is the only contrast agent currently being used to identify liver tumors, hepatic segments, and extrahepatic bile ducts in real time during open and laparoscopic surgery [Jones et al., Eur J Surg Oncol 2017; 43:1622-1627]. This non-specific NIR fluorophore accumulates passively in HCC via the enhanced permeability and retention (EPR) effect [Maeda et al., J Control Release 2000; 65:271-84]. Our results showed that ICG achieves peak uptake over 24 hours post-injection. This time frame is quite long for practical use in the clinic. Moreover, the tumor margins using ICG were indistinct by comparison with that using our NIR-labeled peptide.

Targeted imaging strategies are needed to improve the management of patients with HCC by providing a new approach to detect, characterize, and treat tumors. Current modalities, such as ultrasound (US), computed tomography (CT), magnetic resonance imaging (MRI), and positron emission tomography (PET) cannot effectively determine the benign versus malignant nature of small nodules<2 cm in size [Yu et al., Clin Gastroenterol Hepatol 2011; 9:161-167]. While some progress has been made with serological markers, few advances have been made with tissue markers. Most HCC tumors arise from a background of cirrhosis. Early cancer detection depends on developing a sensitive method that recognizes imaging biomarkers that can distinguish between HCC and non-HCC. Our ex vivo data from WKG*-IRDye800 peptide staining of human HCC with cirrhosis showed high sensitivity and specificity. Thus, a peptide WKG*-IRDye800 that binds specifically to CD44 was identified and validated. This peptide has many properties useful for future clinical translation in management of patients with HCC, including early cancer detection and image-guided surgery.

About 80-90% HCC patients have underlying cirrhosis and effective treatment depends on early recognition of HCC, so developing a sensitive diagnostic approach that can recognize the presence of a suspicious lesion at early stage and differentiate between HCC and non-HCC is the key tasks for imaging. The preclinical data herein support that this peptide can distinguish HCC from cirrhosis with 87% sensitivity and 69% specificity on patient specimens. Since HCC is a highly heterogeneous malignancy in both intratumoral and interpatient manners, targeting the combination of CD44 with other HCC overexpressed biomarkers (for example, GPC3 and/or EpCAM) is also contemplated herein to increase diagnostic efficiency.

In summary, it is contemplated that the CD44-binding peptide WKG* provided herein can be labeled and used clinically for early cancer detection, image-guided resection, and can also be used as a targeting moiety, for example on the surface of nanocarriers for the selective delivery of drug-loaded nanoparticles to CD44-tumors.

### Example 12

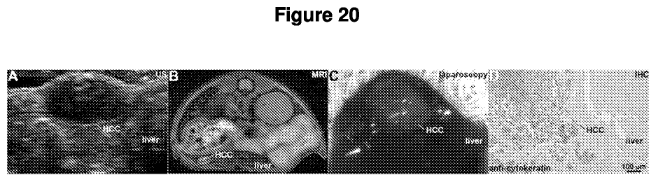

Human Hep3B HCC xenograft tumors were implanted orthotopically in the liver of live nude athymic mice. Animal studies were approved by the University of Michigan University Committee on the Use and Care of Animals (UCUCA). Anesthesia was maintained with inhaled isoflurane. The left lobe of the liver in nude athymic mice at 4-6 weeks of age was injected with a pellet of ˜106 Hep3B cells in 50 μL PBS and Matrigel matrix mixture (1:1) using a 27-gauge needle. The surgical incision was closed with sutures, and the animals were allowed to recover. FIG. 20A-C shows A) Ultrasound (US), B) MRI (9.4T scanner), and C) laparoscopy images from the live mice confirming the orthotopic location of the HCC tumors. The liver was evaluated using immunohistochemistry (IHC), and FIG. 20D shows increased anti-cytokeratin reactivity which confirms the presence of human HCC tumor tissues proliferating within the mouse liver.

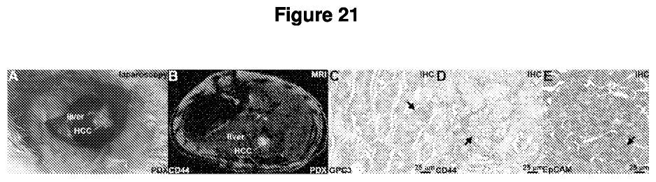

PDTX HCC xenograft tumors were also implanted orthotopically in mice. Fresh HCC specimens were used to develop patient-derived xenograft (PDX) tumors, providing lesions with clinically relevant levels of target expression. Human HCC specimens were implanted subcutaneously first to verify growth, and then orthotopically in liver for MR imaging. NOD Cg-Prkdcll2rgSzJ (NSG) mice were used. These mice carry mutations in severe combined immune deficiency (scid) and a complete null allele of the IL2 receptor common gamma chain (IL2rg^{null}), and are extremely immunodeficient. This model maintains the cellular complexity and architecture from the donor, and mimics the tumor microenvironment with subsequent generations. The tissues were immersed in MACS Tissue Storage Solution (Miltenyi Biotec Inc). After rinsing twice with Hank's Balanced Salt Solution (Thermo Fisher Scientific) in a sterile plate, the tumor was minced using sterile scalpels into small pieces ˜2-3 mm in each dimension. Three pieces of tumor were frozen in liquid nitrogen for RNA/DNA analysis, one piece was processed for routine histology, and two fresh pieces were implanted per animal. The mice were placed in a prone position on the procedure table. A small traverse incision was made on the flank of the mouse. The tumor was inserted into the cavity under skin. The surgical incision was closed with absorbable sutures and wound clips. A control group was injected with a mixture of PBS and Matrigel. Tumor growth was monitored weekly by ultrasound. FIG. 21A shows a representative laparoscopic image.

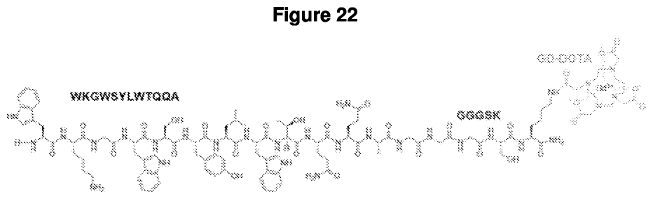

Then, the mice with the PDTX HCC xenograft tumors were injected with Gd-labeled [specifically gadoterate meglumine (Gd-DOTA) labeled] CD44-binding peptide WKG* (FIG. 22) (600 mM in 200 mL PBS). Magnetic resonance (MR) imaging showed PDTX HCC tumor at 1.5 hours post-injection using a 7T scanner, FIG. 21B. A target-to-background (T/B) ratio of 2.68 was measured from the PDTX HCC tumor. Successful tumor implantation in mouse liver was also shown by strong staining for GPC3, CD44, and EpCAM in the resected human HCC specimens using immunohistochemistry (IHC), FIG. 21C-E, respectively.

All documents cited in this application are hereby incorporated by reference in their entirety, with particular attention to the disclosure for which they are referred.

We claim:

1. A reagent comprising a peptide WKGWSYLWTQQA (SEQ ID NO: 1), or a multimer form of the peptide,
wherein the peptide binds to CD44 and
wherein at least one detectable label, at least one therapeutic moiety, or both, are attached to the peptide or a multimer form of the peptide.

2. The reagent of claim 1 comprising at least one detectable label attached to the peptide.

3. The reagent of claim 2 wherein the detectable label is detectable by optical, photoacoustic, ultrasound, positron emission tomography or magnetic resonance imaging.

4. The reagent of claim 3 wherein the label detectable by optical imaging is fluorescein isothiocyanate (FITC).

5. The reagent of claim 3 wherein the label detectable by optical imaging is Cy5.

6. The reagent of claim 3 wherein the label detectable by optical imaging is Cy5.5.

7. The reagent of claim 3 wherein the label detectable by optical imaging is IRdye800.

8. The reagent of claim 3 wherein the label detectable by magnetic resonance imaging is Gd or Gd-DOTA.

9. The reagent of claim 1 wherein the multimer form of the peptide is a dimer formed with an aminohexanoic acid linker.

10. The reagent of claim 2 wherein the detectable label is attached to the peptide by a peptide linker.

11. The reagent of claim 10 wherein a terminal amino acid of the linker is lysine or a cysteine.

12. The reagent of claim 11 wherein the linker comprises the sequence GGGSK set out in SEQ ID NO: 2 or the sequence GGGSC.

13. The reagent of 1, 2, 3, 4, 5, 6, 7, 8, 9, 10, 11 or 12 comprising at least one therapeutic moiety attached to the peptide.

14. The reagent of claim 13 wherein the therapeutic moiety is chemotherapeutic agent.

15. The reagent of claim 13 wherein the therapeutic moiety is a polymeric nanoparticle or micelle.

16. The reagent of claim 14 wherein the micelle is an octadecyl lithocholate micelle.

17. The reagent of claim 16 wherein the nanoparticle or micelle is pegylated.

18. The reagent of claim 14 wherein the nanoparticle or micelle encapsulates carboplatin, paclitaxel, cisplatin, 5-fluorouracil (5-FU), oxaliplatin, capecitabine, irinotecan chlorambucil or sorafenib.

19. A composition comprising the reagent of claim 1, 2, 3, 4, 5, 6, 7, 8, 9, 10, 11, 12, 13, 14, 15, 16, 17 or 18 and a pharmaceutically acceptable excipient.

20. A method for detecting hepatocellular carcinoma cells in a patient comprising the steps of administering the reagent of claim 1, 2, 3, 4, 5, 6, 7, 8, 9, 10, 11 or 12 to the patient and detecting binding of the reagent to hepatocellular carcinoma cells.

21. A method of determining the effectiveness of a treatment for hepatocellular cancer in a patient comprising the step of administering the reagent of claim 1, 2, 3, 4, 5, 6, 7, 8, 9, 10, 11 or 12 to the patient, visualizing a first amount of hepatocellular carcinoma cells labeled with the reagent, and comparing the first amount to a previously-visualized second amount of cells labeled with the reagent,
wherein a decrease in the first amount cells labeled relative to the previously-visualized second amount of cells labeled is indicative of effective treatment.

22. The method of claim 18 further comprising obtaining a biopsy of the cells labeled by the reagent.

23. A method for delivering a therapeutic moiety to hepatocellular carcinoma cells of a patient comprising the step of administering the reagent of claim 13 to the patient.

24. A kit for administering the composition of claim 19 to a patient in need thereof, said kit comprising the composition of claim 19, instructions for use of the composition and a device for administering the composition to the patient.

25. A peptide consisting of the amino acid sequence WKGWSYLWTQQA (SEQ ID NO: 1).
